(19) United States
(12) Patent Application Publication
(10) Pub. No.: US 2019/0002371 A1
(43) Pub. Date: Jan. 3, 2019

(54) DUAL RAFFINATE PARAXYLENE EXTRACTION PROCESS

(71) Applicant: UOP LLC, Des Plaines, IL (US)

(72) Inventors: Linda S. Cheng, Highland Park, IL (US); James A. Johnson, Burr Ridge, IL (US); Bryan K. Glover, Algonquin, IL (US); David S. Krimsky, Cypress, TX (US)

(21) Appl. No.: 16/015,130

(22) Filed: Jun. 21, 2018

Related U.S. Application Data
(60) Provisional application No. 62/527,780, filed on Jun. 30, 2017.

(51) Int. Cl. C07C 5/27

(52) U.S. Cl. CPC C07C 5/2737; C07C 2529/85; C07C 5/2775

(57) ABSTRACT

The present invention relates to heavy desorbent and light desorbent aromatics complex flow scheme. More particularly, this invention relates to the integration of a dual raffinate para-xylene separation process with two isomerization zones. The first isomerization zone is a liquid phase isomerization zone and the second isomerization zone is either an ethylbenzene isomerization zone, or an isomerization zone using MAPSO-31.

### CROSS-REFERENCE TO RELATED APPLICATION

This application claims priority from Provisional Application No. 62/527,780 filed Jun. 30, 2017, the contents of which cited application are hereby incorporated by reference in its entirety.

### FIELD

The present invention relates to heavy desorbent and light desorbent aromatics complex flow scheme. More particularly, this invention relates to the integration of a dual raffinate para-xylene separation process with two isomerization zones. The first isomerization zone is a liquid phase isomerization zone and the second isomerization zone is either an ethylbenzene isomerization zone, or an isomerization zone using MAPSO-31.

### BACKGROUND

In the current heavy desorbent or light desorbent aromatics complex flow scheme, the ethylbenzene conversion and xylene isomerization takes place in the following two types of isomerization processes: ethylbenzene dealkylation isomerization based on an isomerization catalyst, which isomerizes ortho-xylene and meta-xylene to near equilibrium paraxylene to xylene through gas phase xylene isomerization and ethylbenzene dealkylation to benzene and light gas. Second, ethylbenzene isomerization based on an isomerization catalyst, which performs gas phase xylene isomerization and ethylbenzene conversion to xylene via naphthene route liquid phase isomerization, isomerizes ortho-xylene and meta-xylene to near equilibrium paraxylene to xylene in liquid phase, while converting a low level of ethylbenzene through transalkylation. Compared to gas phase isomerization, liquid phase isomar (LPI) has the advantages of lower xylene loss as cracking/ring opening is minimal; lower capital and operating cost as H₂ and associated recycle gas compressor/equipment is not required and liquid phase operation requires less heating/cooling equipment/duty in the PIX loop.

Past work done on the selective ethylbenzene isomerization process investigated a two bed flow scheme, where the first bed was envisioned to be a liquid-phase xylene isomerization reactor followed by a second bed for vapor phase ethylbenzene isomerization. However, there were issues of the first (liquid-phase xylene isomerization) bed heavies hurting stability of the second (vapor-phase ethylbenzene isomerization) bed as the two reactors are arranged in series.

### SUMMARY

The present invention is a process for heavy desorbent and light desorbent aromatics complex flow scheme. More particularly, this invention relates to the integration of a dual raffinate para-xylene separation process with two isomerization zones. The first isomerization zone is a liquid phase isomerization zone and the second isomerization zone is either an ethylbenzene isomerization zone, or an isomerization zone using MAPSO-31.

In the foregoing, all temperatures are set forth in degrees Celsius and, all parts and percentages are by weight, unless otherwise indicated. Other objects, advantages and applications of the present invention will become apparent to those skilled in the art from the following detailed description and drawings. Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

FIG. 1 illustrates a schematic view of the present invention when using a light desorbent.

FIG. 2 illustrates a schematic view of the present invention when using a heavy desorbent.

### DETAILED DESCRIPTION

The present invention can be used for a heavy desorbent and light desorbent aromatics complex flow scheme. More particularly, this invention relates to the integration of a dual raffinate para-xylene separation process with two isomerization zones. The first isomerization zone is a liquid phase isomerization zone and the second isomerization zone is either an ethylbenzene isomerization zone, or an isomerization zone using MAPSO-31.

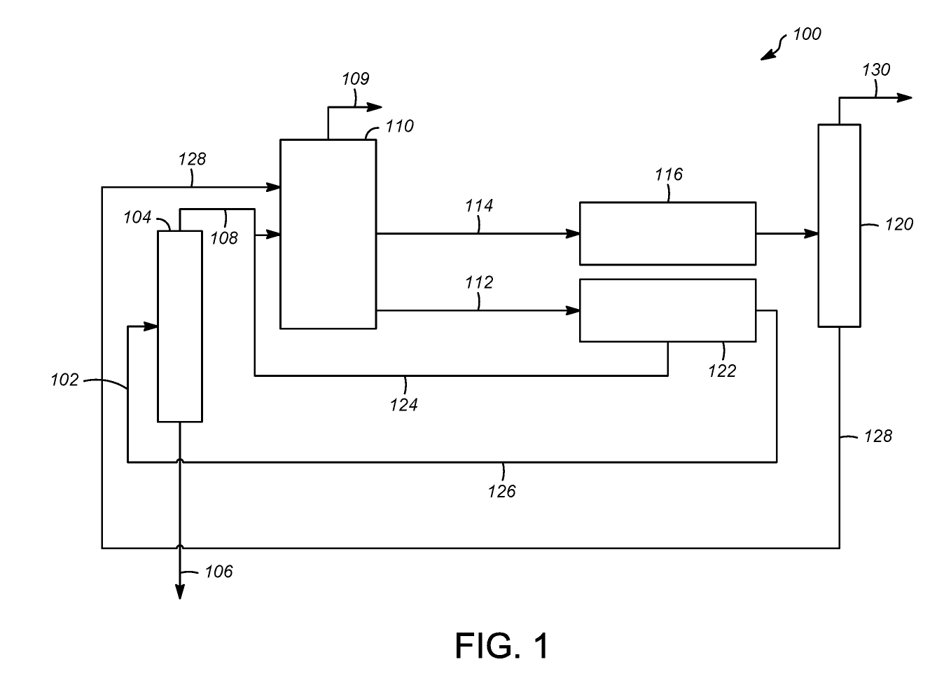

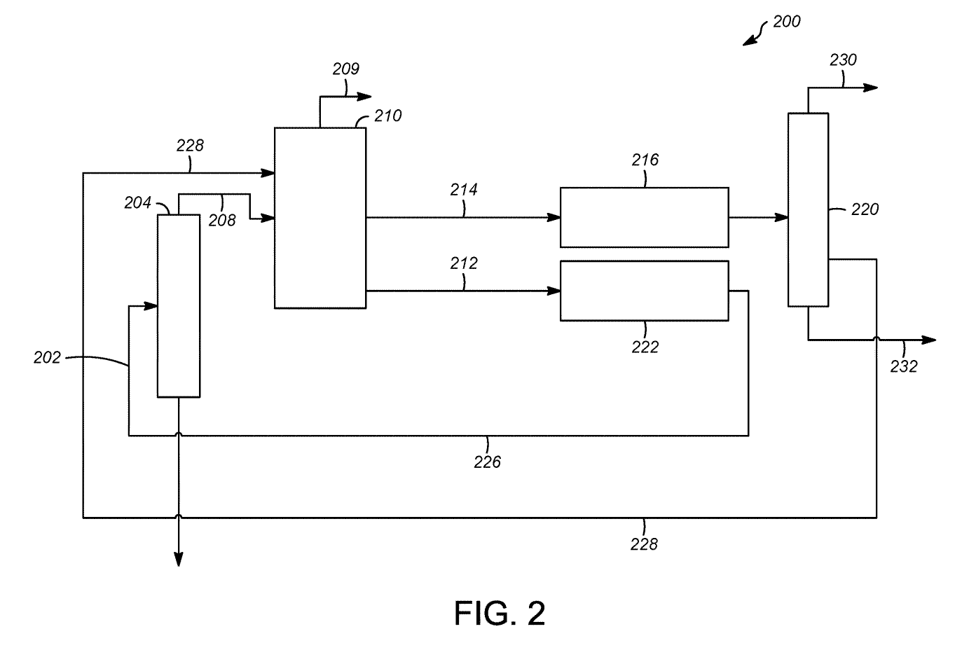

Description of the present invention will be made with respect to FIGS. 1 and 2. The present invention comprises a method and apparatus for the integration of a dual raffinate para-xylene separation process with two isomerization zones.

With respect to the drawings, FIG. 1 depicts an apparatus generally designated as 100 for recovery of para-xylene from a xylene mixture. A feed stream containing a mixture of xylenes, ethylbenzene and heavier aromatics is supplied by line 102 to xylene distillation column 104 which provides a bottoms stream containing heavier aromatics which is withdrawn via line 106. An overhead from xylene column 104 contains xylenes and ethylbenzene and is passed via line 108 to para-xylene separation unit 110. Para-xylene separation unit 110 may be based on a fractional crystallization process or an adsorptive separation process, both of which are well known in the art, and preferably is based on the adsorptive separation process. A para-xylene rich stream is withdrawn from para-xylene separation unit 110 as a first raffinate stream 112 for further product recovery. The para-xylene rich first raffinate stream 112 is sent to an isomerization unit 122. The isomerization unit in FIG. 1 is a liquid phase isomerization unit. The para-xylene rich steam is fractionated to recover desorbent, the ethylbenzene depleted stream containing mainly meta-xylene and ortho-xylene can be sent to a liquid phase isomerization unit for reestablishing xylene equilibrium. Compared to gas phase ethylbenzene dealkylation isomerization unit, liquid phase isomar (LPI) has the advantages of lower xylene loss as cracking/ring opening is minimal; lower capital and operating cost as H₂ and associated recycle gas compressor/equipment is not required and liquid phase operation requires less heating/cooling equipment/duty. When using a light desorbent or a heavy desorbent, the LPI product stream in line 126 is passed back to the xylene column 104. When using a light desorbent, a partial stream of LPI products may be sent directly back to the para-xylene separation until 110 via line 124.

A non-equilibrium mixture of xylenes and ethylbenzene is passed as a second raffinate stream 114 to isomerization reactor assembly 116. Isomerization reactor assembly 116 may contain one or more reaction zones that serve to convert ethylbenzene and isomerize the non-equilibrium mixture of xylenes. The isomerization product is passed via line 118 from isomerization reactor assembly 116 to column 120. The overhead stream 130 comprising mainly C7− is sent to a stabilizer column before passing to an extraction unit and the bottom stream comprising mainly C8+ is sent back to the para-xylene separation unit 110. In one embodiment, the isomerization reactor assembly 116 is an ethylbenzene isomerization unit. In another embodiment, the isomerization reactor assembly 116 is an ethylbenzene dealkylation unit. In another embodiment, the isomerization reactor assembly 116 is a MAPSO-31 unit.

In the first embodiment, where the isomerization reactor assembly 116 is an ethylbenzene isomerization unit, the highly concentrated ethylbenzene stream can be sent to an ethylbenzene isomerization unit based on a commercial catalyst for conversion to xylenes. This will maximize the para-xylene production and minimize the benzene production. The higher the ethylbenzene concentration in the feed, the greater the driving force for ethylbenzene conversion per pass, which will not only reduce costs, but will also result in higher para-xylene product yield due to reduction of C₈ aromatics ring loss.

In the second embodiment, where the isomerization reactor assembly 116 is an ethylbenzene dealkylation unit, the ethylbenzene dealkylation isomerization unit includes ethylbenzene dealkylation catalyst to benzene and light gas.

In the third embodiment, where the isomerization reactor assembly 116 is a MAPSO-31 unit which will convert ethylbenzene to above equilibrium para-xylenes to xylenes. Past work done with MAPSO-31 showed considerable C₈ aromatics loss from ortho-xylene, which will not be the case in this flow scheme as dual raff process can be optimized to minimize ortho-xylene and meta-xylene from the feed going into the MAPSO-31 ethylbenzene isomerization reactor. The above equilibrium para-xylene to xylene can be fed into the para-xylene separation unit to further improve the separation efficiency in a dual feed para-xylene separation unit. This will result in significant further reduction of the recycle traffic in PIX loop as compared to ethylbenzene isomerization.

FIG. 2 depicts an apparatus generally designated as 200 for recovery of para-xylene from a xylene mixture. A feed stream containing a mixture of xylenes, ethylbenzene and heavier aromatics is supplied by line 202 to xylene distillation column 204 which provides a bottoms stream containing heavier aromatics which is withdrawn via line 206. An overhead from xylene column 204 contains xylenes and ethylbenzene and is passed via line 208 to para-xylene separation unit 210. Para-xylene separation unit 210 may be based on a fractional crystallization process or an adsorptive separation process, both of which are well known in the art, and preferably is based on the adsorptive separation process. A para-xylene rich stream is withdrawn from para-xylene separation unit 210 as a first raffinate stream 212 for further product recovery. The para-xylene rich first raffinate stream 212 is sent to an isomerization unit 222. The isomerization unit in FIG. 2 is a liquid phase isomerization unit. The para-xylene rich steam is fractionated to recover desorbent, the ethylbenzene depleted stream containing mainly meta-xylene and ortho-xylene can be sent to a liquid phase isomerization unit for reestablishing xylene equilibrium. Compared to gas phase ethylbenzene dealkylation isomerization unit, liquid phase isomar (LPI) has the advantages of lower xylene loss as cracking/ring opening is minimal; lower capital and operating cost as H₂ and associated recycle gas compressor/equipment is not required and liquid phase operation requires less heating/cooling equipment/duty. The LPI product stream in line 226 is passed back to the xylene column 204.

A non-equilibrium mixture of xylenes and ethylbenzene is passed as a second raffinate stream 214 to isomerization reactor assembly 216. Isomerization reactor assembly 216 may contain one or more reaction zones that serve to convert ethylbenzene and isomerize the non-equilibrium mixture of xylenes. The isomerization product is passed via line 218 from isomerization reactor assembly 216 to column 220. The overhead stream 230 comprising mainly C7− is sent to either the stabilizer column or transalkylation stripper. A side draw 228 is taken from the column 220 and feed the para-xylene separation unit 210. A bottoms stream 232 goes to additional fractionation. For example, the bottoms stream 232 may be passed to an A8 rerun column to recover additional xylenes and to take an additional A9-A10 side draw to a toluene column. The bottoms of the A8 rerun column may then be passed to a heavy aromatics column. In one embodiment, the isomerization reactor assembly 216 is an ethylbenzene isomerization unit. In another embodiment, the isomerization reactor assembly 216 is an ethylbenzene dealkylation unit. In another embodiment, the isomerization reactor assembly 216 is a MAPSO-31 unit.

In the first embodiment, where the isomerization reactor assembly 216 is an ethylbenzene isomerization unit, the highly concentrated ethylbenzene stream can be sent to an ethylbenzene isomerization unit based on a commercial catalyst for conversion to xylenes. This will maximize the para-xylene production and minimize the benzene production. The higher the ethylbenzene concentration in the feed, the greater the driving force for ethylbenzene conversion per pass, which will not only reduce costs, but will also result in higher para-xylene product yield due to reduction of C8 aromatics ring loss.

In the second embodiment, where the isomerization reactor assembly 216 is an ethylbenzene dealkylation unit, the ethylbenzene dealkylation isomerization unit includes an ethylbenzene dealkylation catalyst to dealkylate ethylbenzene to benzene and light gas.

In the third embodiment, where the isomerization reactor assembly 216 is a MAPSO-31 unit which will convert ethylbenzene to above equilibrium para-xylenes to xylenes. Past work done with MAPSO-31 showed considerable C₈ aromatics loss from ortho-xylene, which will not be the case in this flow scheme as dual raff process can be optimized to minimize ortho-xylene to meta-xylene from the feed going into the MAPSO-31 ethylbenzene isomerization reactor. The above equilibrium para-xylene to xylene can be fed in zone II to further improve the separation efficiency in a dual feed para-xylene separation unit. This will result in significant further reduction of the recycle traffic in PIX loop as compared to ethylbenzene isomerization.

### SPECIFIC EMBODIMENTS

While the following is described in conjunction with specific embodiments, it will be understood that this description is intended to illustrate and not limit the scope of the preceding description and the appended claims.

A first embodiment of the invention is a process for hydrocarbon conversion comprising passing a feed stream to a para-xylene separation zone to produce a first raffinate stream and a second raffinate stream; passing the first raffinate stream to a first isomerization zone to produce a first isomerization zone product stream; and passing the second raffinate stream to a second isomerization zone to produce a second isomerization zone product stream. An embodiment of the invention is one, any or all of prior embodiments in this paragraph up through the first embodiment in this paragraph, wherein the hydrocarbon conversion process uses a heavy desorbent. An embodiment of the invention is one, any or all of prior embodiments in this paragraph up through the first embodiment in this paragraph, wherein the hydrocarbon conversion process uses a light desorbent. An embodiment of the invention is one, any or all of prior embodiments in this paragraph up through the first embodiment in this paragraph, wherein the first isomerization zone is a liquid phase isomerization zone. An embodiment of the invention is one, any or all of prior embodiments in this paragraph up through the first embodiment in this paragraph, wherein the second isomerization zone is an ethylbenzene isomerization zone. An embodiment of the invention is one, any or all of prior embodiments in this paragraph up through the first embodiment in this paragraph, wherein the ethylbenzene isomerization zone comprises an ethylbenzene dealkylation catalyst. An embodiment of the invention is one, any or all of prior embodiments in this paragraph up through the first embodiment in this paragraph, wherein the ethylbenzene isomerization zone comprises a MAPSO-31 catalyst. An embodiment of the invention is one, any or all of prior embodiments in this paragraph up through the first embodiment in this paragraph, wherein the second isomerization zone is an ethylbenzene dealkylation zone.

A second embodiment of the invention is a process for hydrocarbon conversion comprising passing a feed stream to a para-xylene separation zone using a heavy desorbent to produce a first raffinate stream and a second raffinate stream; passing the first raffinate stream to a liquid phase isomerization zone to produce a first isomerization zone product stream; and passing the second raffinate stream to a ethylbenzene isomerization zone to produce a second isomerization zone product stream. An embodiment of the invention is one, any or all of prior embodiments in this paragraph up through the second embodiment in this paragraph, wherein the ethylbenzene isomerization zone comprises an ethylbenzene dealkylation catalyst. An embodiment of the invention is one, any or all of prior embodiments in this paragraph up through the second embodiment in this paragraph, wherein the ethylbenzene isomerization zone comprises a MAPSO-31 catalyst.

A third embodiment of the invention is a process for hydrocarbon conversion comprising passing a feed stream to a para-xylene separation zone using a light desorbent to produce a first raffinate stream and a second raffinate stream; passing the first raffinate stream to a liquid phase isomerization zone to produce a first isomerization zone product stream; and passing the second raffinate stream to a second isomerization zone to produce a second isomerization zone product stream. An embodiment of the invention is one, any or all of prior embodiments in this paragraph up through the third embodiment in this paragraph, wherein the second isomerization zone is an ethylbenzene isomerization zone. An embodiment of the invention is one, any or all of prior embodiments in this paragraph up through the third embodiment in this paragraph, wherein the ethylbenzene isomerization zone comprises an ethylbenzene dealkylation catalyst. An embodiment of the invention is one, any or all of prior embodiments in this paragraph up through the third embodiment in this paragraph, wherein the ethylbenzene isomerization zone comprises a MAPSO-31 catalyst. An embodiment of the invention is one, any or all of prior embodiments in this paragraph up through the third embodiment in this paragraph, wherein the second isomerization zone is an ethylbenzene dealkylation zone. An embodiment of the invention is one, any or all of prior embodiments in this paragraph up through the third embodiment in this paragraph, wherein the ethylbenzene dealkylation zone comprises an ethylbenzene dealkylation catalyst.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for hydrocarbon conversion comprising:
passing a feed stream to a para-xylene separation zone to produce a first raffinate stream and a second raffinate stream;
passing the first raffinate stream to a first isomerization zone to produce a first isomerization zone product stream; and
passing the second raffinate stream to a second isomerization zone to produce a second isomerization zone product stream.

2. The process of claim 1, wherein the hydrocarbon conversion process uses a heavy desorbent.

3. The process of claim 1, wherein the hydrocarbon conversion process uses a light desorbent.

4. The process of claim 1, wherein the first isomerization zone is a liquid phase isomerization zone.

5. The process of claim 1, wherein the second isomerization zone is an ethylbenzene isomerization zone.

6. The process of claim 5, wherein the ethylbenzene isomerization zone comprises an ethylbenzene dealkylation catalyst.

7. The process of claim 5, wherein the ethylbenzene isomerization zone comprises a MAPSO-31 catalyst.

8. The process of claim 1, wherein the second isomerization zone is an ethylbenzene dealkylation zone.

9. A process for hydrocarbon conversion comprising:
passing a feed stream to a para-xylene separation zone using a heavy desorbent to produce a first raffinate stream and a second raffinate stream;
passing the first raffinate stream to a liquid phase isomerization zone to produce a first isomerization zone product stream; and
passing the second raffinate stream to a ethylbenzene isomerization zone to produce a second isomerization zone product stream.

10. The process of claim 9, wherein the ethylbenzene isomerization zone comprises an ethylbenzene dealkylation catalyst.

11. The process of claim 9, wherein the ethylbenzene isomerization zone comprises a MAPSO-31 catalyst.

12. A process for hydrocarbon conversion comprising:
passing a feed stream to a para-xylene separation zone using a light desorbent to produce a first raffinate stream and a second raffinate stream;
passing the first raffinate stream to a liquid phase isomerization zone to produce a first isomerization zone product stream; and
passing the second raffinate stream to a second isomerization zone to produce a second isomerization zone product stream.

13. The process of claim 12, wherein the second isomerization zone is an ethylbenzene isomerization zone.

14. The process of claim 12, wherein the ethylbenzene isomerization zone comprises an ethylbenzene dealkylation catalyst.

15. The process of claim 12, wherein the ethylbenzene isomerization zone comprises a MAPSO-31 catalyst.

16. The process of claim 12, wherein the second isomerization zone is an ethylbenzene dealkylation zone.

17. The process of claim 16, wherein the ethylbenzene dealkylation zone comprises an ethylbenzene dealkylation catalyst.
